(19) United States
(12) Patent Application Publication
(10) Pub. No.: US 2004/0092920 A1
(43) Pub. Date: May 13, 2004

(54) Cutting and removal of biologic tissue by pressurized propulsion of ice particles

(76) Inventor: Eyal Rozenshpeer, Tel Aviv (IL)

Correspondence Address: Albert Wai Kit Chan, Law Offices of Albert Wai Kit Chan, World Plaza Suite 604, 141 07 20th Avenue, Whitestone, NY, 11357, US

(21) Appl. No.: 10/466,499

(22) PCT Filed: Dec. 20, 2001
(86) PCT No.: PCT/US01/48772

(51) Int. Cl. A61B 18/02

(52) U.S. Cl. 606/22

(57) ABSTRACT

An apparatus and methods for cutting and removal of biological tissue using pressurized propulsion of ice particles is disclosed. An apparatus for cutting and removal of biological tissue includes: an ice particle generator, for producing ice particles; a particle delivery element, connected to the ice particle generator, for transporting the ice particles from the ice particle generator; an injection handpiece, connected to the particle delivery element, the injection handpiece having an injection outlet; and, a high pressure source, connected to the injection handpiece, for propelling the ice particles in a jet stream of fluid from the injection outlet, under high pressure and at high linear velocity, so as to cut and remove a desired portion of the biological tissue.

### FIELD AND BACKGROUND OF THE INVENTION

The present invention relates to an apparatus and methods for cutting and removal of biological tissue and, more particularly, to an apparatus and methods for cutting, removal and debridement of biological tissue using pressurized propulsion of ice particles.

Many circumstances require the removal of a portion of unwanted, perhaps damaged or diseased, biological tissue without damage to surrounding desirable, generally healthy tissue. Treatment of burns, debridement of necrotic tissue such as that of necrotic pressure (decubitus) ulcers or skin peeling for treatment of scars, photoaged skin or removal of tattoos are just some examples.

A burn injury is one of the most devastating injuries a person can suffer. Over 2 million burns require medical attention each year in the USA, with 14,000 deaths resulting. In a third-degree, or full thickness, burn all epithelial elements are destroyed, leaving no potential for re-epitheliazation. These burns have a characteristic white, waxy appearance and leathery texture. When 40% or more of the body surface is burned, a hypermetabolic state, electrolyte imbalance, hypothermia, profound catabolic state, infection, sepsis, and multi-organ failure are natural consequences. Burn wound inflammation even in the absence of infection can result in organ dysfunction and perpetuation of the hypermetabolic state due to the release of toxins and inflammatory mediators from the necrotic tissue.

The ultimate remedy is to restore the natural barrier of the body by closing the wound with a skin graft. Skin grafts are thin meshed split skin, which depend on the blood and oxygen supply from the tissue underneath. Any amount of dead, necrotic tissue will prevent graft implantation. Therefore, the centerpiece of modern burn care involves the prompt removal of necrotic tissue with immediate biologic closure. Early burn wound excision and closure is now widely practiced in North America and is typically carried out as a series of staged excisions of all deep and full thickness components of the wound over the fist post-injury week The current method for debridement involves excision of the necrotic tissue using a knife. The tissues are softened prior to excision through use of a wet dressing or by soaking and scrubbing, often with a sponge, and often in a bath or shower. In order to achieve clean tissue suitable for grafting repeated use of these measures is usually required. There are many problems and shortcomings associated with the use of this process. These patients are very prone to hypothermia: long exposure even to room temperature can drop the core temperature seriously. The process is typically quite time-consuming and very painful. Some healthy tissue is generally also excised along with the necrotic tissue.

Debridement of necrotic tissue is also a fundamental component of care for decubitus ulcers or pressure sores. Pressure sores develop as local pressure is applied to the tissues for a long time, generally hours, reducing the blood supply to the tissue and causing ischemic insult. When this process extends over longer periods of time, the insult becomes irreversible, resulting in deeper and larger areas of necrotic tissue, which can involve the skin, underlying fat, fascia, muscle and even bone. These sores, also known as pressure ulcers, or bed sores, are associated with immobility, poor nutrition and aging and are prevalent among patients with neurologic disability, diabetes and the elderly. Over 2% of the population of the United States is affected by these sores and the cost of their treatment is currently estimated to be as high as 8.5 billion dollars. The sores typically produce a foul odor as result of the necrotic tissue and colonization with anaerobic bacteria. This is a problem both for the care providers who must work in its presence, and for the patient, who suffers embarrassment because of this. Abscesses in the sores release toxins and inflammatory mediators, which result in systemic illness, fever, weight loss, and flu-like symptoms.

Treatment of pressure ulcers involves mobilization of the patient, debridement of the necrotic tissue, and in cases involving large and deep wounds, surgical closure. Debridement of the necrotic tissue is a necessary and critical part of the management, and can be accomplished in a number of ways including sharp mechanical excision using the surgical scalpel or irrigation and dressing with antiseptics and necrotic tissue softening agents. Debridement must be repeated a number of times as the area of necrosis spreads. These methods are subject to similar deficiencies as detailed above for the treatment of burns. For example, surgical excision of the necrotic tissue, if done properly, usually includes removing a healthy rim of skin as well as underlying tissues such as fascia, muscle, fat, tendon, etc. Because of the complexity of the circumstances, process and its preparations; affected patients who often have anemia and a bleeding tendency; lack of post-operative care; and a conventional bedside procedure that is sub-optimal; together with the unpleasant odor; health care staff tend to work as fast as they can, and often superficially, leaving some necrotic tissue in the wound. This leads to longer recovery periods and huge expenses for hospitalization and treatments.

In other circumstances unwanted cutaneous tissues are removed, often to allow healthier appearing skin to regenerate. For generally aesthetic reasons it may be desired to remove unsightly scars (which may result from surgery, injury or acne or chicken pox) or tattoos. Aesthetic surgeons also typically use processes for treating wrinkles or pigmentary irregularities and aged and photodamaged skin by destroying the superficial outer layers of the skin and allowing the skin to subsequently regenerate, leaving skin with a younger rejuvenated appearance. This has been accomplished in several ways: typically using chemical peels, laser or dermabrasion. Chemical peeling induces itself a controlled, partial-thickness chemical burn of the epidermis and the outer dermis. Various techniques are available to regulate the depth of the burn. Following this induced wound, reepitheliazation through regeneration of peeled skin from follicular and eccrine duct epithelium results in a fresh, organized epidermis. With deeper burns with regeneration of the dermis with orderly, compact collagen wrinkles can be removed. Chemical agents such as 10-25% TCA, alpha hydroxy acids, such as glycolic acid, and retinoids are used for superficial peels; TCA, 35-50%, is usually used for medium depth peels; and phenol for deep peels. These peeling techniques suffer from the limitations resulting from the toxic and systemic effects of these chemical agents (for, example, cardiotoxicity of phenol), allergic reactions, consistent erythema, scarring and pain.

Alternatives to the use of chemical peels include the use of the CO₂ laser or dermabrasive resurfacing. In dermabrasion, the epidermis and dermis are abraded, or planed, generally with the use of a rapidly rotating wire brush or diamond fraise. The diamond fraise is spun at high speeds and drawn over the skin surface so that the entire epidermis and upper dermis are removed. The sweat glands and hair follicles remain and proliferate to re-epithelialize the now smooth-planed skin surface. Other dermabrasive techniques use an apparatus to deliver a flow of air and reducing substances (that is abrasive particles such as corundum crystals) to effect the skin abrasion “microdermabrassion”. (see for example U.S. Pat. No. 5,037,432 to Molinari, U.S. Pat. Nos. 5,100,412 to Rosso and 5,810,842 to Di Fiore et. al.) All of these devices and methods suffer from the deficiencies that the sand-like abrasive particles can easily get into the tissue and lo cause foreign body reactions. They require a vacuum system for collecting the used abrasive particles, which can not be re-used as they are contaminated with biologic materials and which therefore must be disposed of properly. For these reasons the potency of this method remains low and it is used generally only within cosmetic parameters (e.g., with low power).

Following dermabrasion the patient experiences discomforts such as a deep warmth and throbbing sensation as a result of an induced inflammatory process. The skin can be treated with dry ice (CO₂) prior to mechanical dermabrasion or chemical peel as well. Cold injury can further damage the skin surface and can create skin turgor that enhances the dermabrasion.

Jets of fluid under high pressure have been used for fragmenting and removing diseased tissue (see for example U.S. Pat. Nos. 4,560,373 to Sugino et. al., 4,913,698 to Ito et. al. and 5,037;431 to Summers et. al.) All these techniques suffer from the deficiencies that enormously high pressures are used to fragment the tissue. This magnitude of pressure can create dissection between the tissues and create complications and destroy healthy tissue.

The approaches to dermabrasion using air driven corundum crystals resembles industrial “sand blasting” techniques used for such applications as cleaning, polishing, decontamination, or paint removal of surfaces such as walls or floors. Industrial applications of this sort have also been accomplished using a technique that has been termed “ice blasting” or “cryogenic blasting”. Particles (pellets) of (frozen water) ice or dry ice (CO₂) are sprayed as an abrasive by a jet of pressurized air. This has been used for applications such as removing rust soot, carbon particles, or paint from engine or heavy machinery parts, or for cleaning buildings or marine vessels or for nuclear decontamination. It has the advantage that the abrasive material melts upon impact or shortly thereafter, simplifying the disposal process, and the melting ice also washes away the removed material creating a “scrub and wash effect”. In addition, use of ice pellets as an abrasive has been found to be more suitable when maintenance of dimensional stability is critical, when degradation of the product's finish is a concern, or when the substrate is delicate or thin. The technology employed is the subject of a number of patents (including for example, U.S. Pat. No. 5,785,581 to Settles, U.S. Pat. No. 6,001,000 to Visaisouk et al., PCT Publication No. WO 94/23895, PCT Publication No. WO 94/16861, PCT Publication No. WO 96/35913, and PCT Publication No. WO 98/36230) and commercial ice blasting systems are available for industrial applications. The ice particles used are very large (up to 2 cm.) and under very high pressure (150-220 psi). None of the previously disclosed systems of the prior art have been proposed for use in biological applications nor are they suitable for such use due to limitations such as the size and structure of the delivery systems, size of the pellets used, temperature control, and magnitude of the pressures and low temperature used.

There is thus a widely recognized need for, and it would be highly advantageous to have a system and a method for cutting and removal of biologic tissue by pressurized propulsion of ice particles, devoid of the above limitations.

### SUMMARY OF THE INVENTION

According to the present invention there is provided a system and a method that can be used for cutting and removal of biological tissue. Specifically, the present invention can be used to cut, remove and debride biological tissue using pressurized propulsion of ice particles.

According to one aspect of the present invention there is provided an apparatus for cutting and removal of biological tissue, which includes: an ice particle generator, for producing ice particles; a particle delivery element, connected to the ice particle generator, for transporting the ice particles from the ice particle generator, an injection handpiece, connected to the particle delivery element, the injection handpiece having an injection outlet; and, a high pressure source, connected to the injection handpiece, for propelling the ice particles in a jet stream of fluid from the injection outlet, under high pressure and at high linear velocity, so as to cut and remove a desired portion of the biological tissue.

According to another aspect of the present invention there is provided a method for cutting and removal of biological tissue which includes the steps of: generating ice particles of a predetermined and appropriate size, delivering the ice particles to an injection handpiece, and, propelling the ice particles toward the biological tissue in a jet stream of a predetermined and appropriate high speed and linear velocity, so as to effect cutting and removal of a desired portion of the biological tissue.

According to yet another aspect of the present invention there is provided a method for cutting and removal of biological tissue including the steps of: providing an apparatus for cutting and removal of the biological tissue; adjusting at least one parameter of the apparatus; operatively engaging the apparatus so as to produce a jet of ice particles; directing the jet of propelled ice particles to impact on the biological tissue to be removed, at such an angle to the tissue that a desired portion of the tissue and only the desired portion of the tissue is removed; and, mechanically moving the jet to change a point of impact so as to remove all of, and only, the desired portion of the tissue.

According to further features in preferred embodiments of the invention described below, the composition of the ice particles is selected from the group consisting of frozen water, frozen saline, and solidified carbon dioxide. According to still further features in the described preferred embodiments the ice particles are composed of a frozen solution of a diluent containing a chemical. According to still further features in the described preferred embodiments, the diluent is selected from the group consisting of water and saline. According to still further features in the described preferred embodiments, the chemical is selected from the group consisting of an antibiotic, an antiseptic, an analgesic, a local anesthetic, an anticoagulant, and a growth factor.

According to still further features in the described preferred embodiments, the apparatus further includes a low-pressure compressor for moving the ice particles from the ice particle generator into the particle delivery element.

According to still further features in the described preferred embodiments the ice particles are sucked from the particle delivery element into the injection handpiece by venturi effect.

According to still further features in the described preferred embodiments the particle delivery element is a cannula According to still further features in the described preferred embodiments, the cannula is transparent. According to still further features in the described preferred embodiments, the cannula is fabricated from a flexible material. According to still further features in the described preferred embodiments, the cannula has an inner lumen, the inner lumen being coated with a material to prevent adherence of the ice particles to the cannula.

According to still further features in the described preferred embodiments, the apparatus further includes a heater element for preventing the ice particles from aggregating and obstructing movement through the particle delivery element According to still further features in the described preferred embodiments the heater element includes a wire, the wire being constructed from an electrically resistive material, the wire circumferentially surrounding the particle delivery element so as to apply heat to the particle delivery element. According to still further features in the described preferred embodiments the wire is connected to a heater control so as to maintain the applied heat at a desired temperature.

According to still further features in the described preferred embodiments the high pressure source has a variable output pressure. According to still further features in the described preferred embodiments the output pressure is between 10 psi and 100 psi. According to still further features in the described preferred embodiments the output pressure is between 20 psi and 80 psi.

According to still further features in the described preferred embodiments the high pressure source propels the ice particles in a stream of gas. According to still further features in the described preferred embodiments the gas is compressed air. According to still further features in the described preferred embodiments the stream of gas is maintained at a temperature of between −1 and 0 degrees Celsius. According to still further features in the described preferred embodiments the high pressure source propels the ice particles in a stream of liquid.

According to still further features in the described preferred embodiments the apparatus further includes a control switch for actuating the high pressure source. According to still further features in the described preferred embodiments the control switch is a footswitch.

According to still further features in the described preferred embodiments the high pressure source is configured to be operable to continuously propel the ice particles. According to still further features in the described preferred embodiments the high-pressure source is configured to be operable to propel the ice particles in pulses. According to still further features in the described preferred embodiments the pulses are between 4 and 15 seconds in duration.

According to still further features in the described preferred embodiments the injection outlet is between 5 mm and 50 mm in diameter.

According to still further features in the described preferred embodiments the apparatus further includes a suction mechanism for aspirating melting ice particles and removed tissue fragments. According to still further features in the described preferred embodiments the suction mechanism includes a suction nozzle. According to still further features in the described preferred embodiments the suction nozzle is shaped as a conical dome, the dome having an elastic lip on the distal edge of the dome that conforms to a contour of a surface against which the dome is applied. According to still further features in the described preferred embodiments, the dome is transparent.

According to still further features in the described preferred embodiments, the suction nozzle is a suction hood having a conical dome at a distal end of the hood, the dome having an elastic lip on a distal edge of the dome that conforms to a contour of a surface against which the dome is applied, the hood having a flexible neck portion at a proximal end of the hood.

According to still further features in the described preferred embodiments, the injection handpiece and the suction hood are so configured as to place the injection handpiece within the flexible neck of the suction hood. According to still further features in the described preferred embodiments the suction hood further includes a one way valve configured so as to be operable to permit air entry.

According to still further features in the described preferred embodiments, the suction mechanism further includes a collection container for collecting the melting ice particles and the removed tissue fragments.

According to still further features in the described preferred embodiments the ice particle generator includes a sizer mechanism to insure that all of the ice particles are of a predetermined and uniform size. According to still further features in the described preferred embodiments the predetermined size is between 0.001 mm and 50 mm. According to still further features in the described preferred embodiments the predetermined size is between 0.001 mm and 15 mm. According to still further features in the described preferred embodiments the predetermined size is between 0.001 mm and 6 mm.

According to still further features in the described preferred embodiments the apparatus further includes a central processor mechanism for control of at least one parameter of operation of the apparatus. According to still further features in the described preferred embodiments the central processor mechanism is programmable. According to still further features in the described preferred embodiments the at least one parameter of operation is selected from the group consisting of temperature of the fluid stream, pressure of the fluid stream, velocity of propulsion of the fluid steam, temperature of the ice particles, and size of the ice particles.

According to still further features in the described preferred embodiments the apparatus is configured for cutting and removal of human tissue.

According to still further features in the described preferred embodiments the apparatus is configured for cutting and removal of cutaneous tissue.

According to still further features in the described preferred embodiments the apparatus is configured for cutting and removal of necrotic tissue.

According to still further features in the described preferred embodiments the apparatus is configured for debriding a burn.

According to still further features in the described preferred embodiments the apparatus is configured for debriding a pressure sore.

According to still further features in the described preferred embodiments the apparatus is, configured for performing a skin peel.

According to still further features in the described preferred embodiments, in the method described hereinabove, the at least one parameter of operation is selected from the group consisting of: size of an injection outlet, size of the ice particles, pressure of the jet, velocity of the jet, pulsation of the jet, duration of the pulsation, and length of time of treatment

According to still further features in the described preferred embodiments the method further includes the step of: aspirating and collecting the ice particles and water generated from melting of the ice particles along with fragments of the tissue removed.

According to still further features in the described preferred embodiments the method father includes the steps of: placing a suction hood over an area of the tissue to be removed; applying light pressure to the suction hood to create a tight seal; and, holding an injection handpiece; before the step of operatively engaging the apparatus so as to produce a jet of ice particles.

According to still further features in the described preferred embodiments the method further includes the initial step of applying a topical chemical agent to the tissue to disinfect and color an area designated for treatment.

The present invention successfully addresses the shortcomings of the presently known configurations by providing a system and method for cutting, removal and debridement of biological tissue using pressurized propulsion of ice particles. The present invention provides an analgesic and anaesthetic effect, so that the need for general or additional local anesthesia is reduced or eliminated. It provides an irrigation effect that maintains a sterile field and permits a clear view of the depth of tissue removal achieved in real time. It allows control of depth of tissue removal with a wide margin of safety, in real time, and by modification of simple parameters. No active chemicals are required, reducing the risk of hypersensitivity reactions or systemic effects. The cooling effect of the ice particles further provides as well an anti-inflammatory effect The cooling of the skin raises skin turgor making the skin more amenable to mechanical abrasion. Debridement can be easily limited to only necrotic tissues if desired. Use of a closed system protects the operator from infectious material and the unpleasant odor that accompany necrotic lesions. The ability to thoroughly debride, under pressure, material at the bottom of necrotic craters, along with the irrigation effect, allows improved drainage of infected abscesses and relief from the systemic inflammatory response that accompany these abscesses. Simultaneous irrigation and debridement shortens the process of care, saving money and reducing both the human resources required as well as expensive dressings and the like.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is diagram of a first embodiment of a system for cutting and removal of biologic tissue by pressurized propulsion of ice particles;

FIG. 2 is a diagram of an alternate preferred embodiment of a system for cutting and removal of biologic tissue by pressurized propulsion of ice particles according to the present invention, illustrating a collection system;

FIG. 3 is a diagram of an alternate preferred embodiment of a system for cutting and removal of biologic tissue by pressurized propulsion of ice particles according to the present invention, illustrating a closed system where the injection nozzle and collection system are seated within a suction hood;

FIG. 4 is a schematic flow diagram illustrating the steps in a preferred embodiment of the method for cutting and removal of biologic tissue by pressurized propulsion of ice particles using preferred embodiments of the apparatus of the present invention;

FIG. 5 is a schematic flow diagram illustrating the steps in an alternate preferred embodiment of the method for cutting and removal of biologic tissue by pressurized propulsion of ice particles using the apparatus of the present invention for use for debridement of necrotic tissue; and,

FIG. 6 is a schematic flow diagram illustrating the steps in an alternate preferred embodiment of the method for cutting and removal of biologic tissue by pressurized propulsion of ice particles of the present invention when used for dermabrasion.

### DESCRIPTION OF THE PREFERRED EMBODIMENTS

The present invention is of an apparatus that can be used for cutting and removal of biological tissue. Specifically, the present invention can be used to cut, remove and debride biological tissue using pressurized propulsion of ice particles. The present invention further discloses a method for use of such an apparatus.

The principles and operation of an apparatus for cutting, removal and debridement of biological tissue using pressurized propulsion of ice particles according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

For purposes of this specification and the accompanying claims, the term “biological tissues” should not be seen as limiting to only human tissues, but also to include tissues of other species. The invention is described hereinbelow in conjunction with specific embodiments thereof and with reference to specific examples to illustrate the invention in a non-limiting fashion. Although the present invention is described hereinbelow in conjunction with specific embodiments thereof and with specific examples of its use in specific medical and surgical applications involving the skin and supporting tissues and structures, to illustrate the invention in a non-limiting fashion, it is not intended that the present invention be limited to use in cutaneous tissues (skin), human tissues or to medical or surgical applications. Specifically envisioned as being encompassed by the present invention are also such applications as removing the scales from fish or feathers from chickens; cleaning and decontamination of mustard gas or other chemical warfare agents from the skin or as a substitute for emergency decontamination showers found in hospitals for hazardous chemical exposure; for surgical scrubbing of hands prior to an operation or for cleaning and scrubbing in preparation of an operative field; thinning out fat tissue; thinning out thick muscle tissue flaps; debulking tumors, especially those with finger-like projections; removing necrotic tissue in orthopedic operations, removing dental plaque and is calculus and treating gingival and periodontal disease; and removing atheromatous plaque and calcification or thrombi from blood vessels as further non-limiting examples.

For purposes of this specification and the accompanying claims, the terms “ice pellets,” “ice particles,” “ice particulates,” and “ice grains” are used interchangeably to refer to a small body, mass or piece of solidified frozen (sterile) water, (sterile) saline or other liquid, including frozen solutions consisting of a diluent such as saline or water with another chemical such as antibiotics, antiseptics, growth factors, local anesthetics, analgesics, anticoagulants, and the like. It also includes small pieces of any substance resembling frozen water, that is, the frozen state of other substances usually found as a gas or liquid, such as solidified carbon dioxide. Further it also is meant to encompass suspensions of small pieces of frozen fluid in a liquid medium.

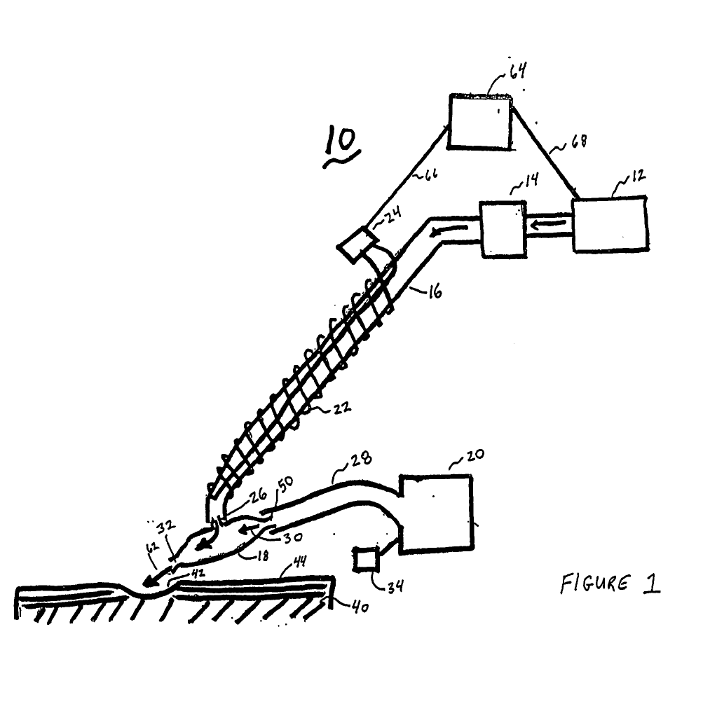

Referring now to the drawings, FIG. 1 illustrates a preferred embodiment of the present invention, an apparatus 10 for cutting, removal and debridement of biological tissue using pressurized propulsion of ice particles. Apparatus 10 includes an ice pellet generator (12), a low-pressure compressor (14), a feeding cannula (16), an injection handpiece (18), and a high pressure compressor (20). The ice pellets that are formed in the ice pellet generator are delivered to the feeding cannula by the low-pressure compressor. Then they are sucked from the feeding cannula into the injection handpiece by venturi effect.

The ice pellet generator (12) serves to create particles of ice of a predetermined and homogeneous size. The generator serves to solidify water into ice, separate ice particles from snow and water, size the particles (i.e., insure that the particles are of the predetermined and uniform size) and transport them to the feeding cannula The particles are propelled into the feeding cannula by the low-pressure compressor by a dry and cold (−6 to −10 degrees Celsius) stream of air at low (4-10 psi) pressure.

The ice pellets can be made in any one of several ways and one of ordinary skills in the art would know how to operatively assemble such a device from commercially available components or purchase and modify a commercially available device. Examples of the well known and commercially available [for example, those marketed by Universal Ice Blast, Inc. of Kirkland, Wash.] prior art technologies that can be used to produce ice pellets include, but are not limited to, those that involve either scraping and/or harvesting or methods involving grinding or crushing. In the first method, water is sprayed on a cold and rotating drum to form a uniform thin layer of ice. A fixed knife is positioned parallel to the rotating drum. The knife cuts ice particles of homogeneous and predetermined size. In a second method, water is frozen into a block of ice and then crushed into particles of desired dimensions. An artificial nucleator can also be added to the water to raise the static freezing temperature. As discussed hereinbelow, ice pellets of various dimensions can be generated for different purposes. In general, smaller particles have a very short contact time before phase change occurs which tends to generate maximum tensile force more superficially. One of ordinary skill in the art will be able to include mechanisms for sizing and separation of the ice particles in ice pellet generator 12.

Ice particles are transported from ice pellet generator 12 by the low-pressure flow into feeding cannula 16. Feeding cannula 16 is preferably fabricated from a transparent and flexible material, including for example, but not limited to, a polyvinyl chloride. The inner lumen of the tube is coated with a low friction coefficient material, such as polytetrafluoroethylene (Teflon™), for example, to prevent adherence of the ice particles to the cannula. A metal, (or other electrically resistive material, such as metal alloy), preferably tungsten, wire 22 is wound spirally over feeding cannula 16 forming a coil that serves as a heater element that prevents the ice particles from aggregating and blocking the outflow through feeding cannula 16. The wire (22) is connected to heater control 24 which includes a thermostat and which maintains an electrical current through wire 22 in a manner to keep the heat applied to the cannula at the desired temperature.

Feeding cannula 16 is attached to a particle intake valve 26 on injection handpiece 18. As described hereinabove, the ice particles are pulled into injection handpiece 18 through particle intake valve 26 by a venturi effect generated suction. The venturi flow is created by a high pressure stream of air (30) through the lumen of injection handpiece 18. This high pressure flow is created by high pressure compressor 20 which has a variable output pressure and which is flow is connected by appropriate flexible tubing (28) to the high pressure intake opening at one end of injection handpiece 18. The high pressure main stream of air (30) is maintained at a temperature just below the freezing point (between −1 and 0 degrees C.). At this temperature, as the particles move laterally during their phase change from a solid to a liquid, the ice pellets have maximal abrasive ability. The airflow generated by the high pressure imparts a high kinetic energy to the ice particles for a stronger power of abrasion.

In alternate preferred embodiments of the system of the present invention, the high-pressure compressor (20) serves to produce a high-pressure flow of a fluid substance other than compressed air. Such fluids include, as non-limiting examples, other gases, such as oxygen mixtures, as well as liquids, including water, saline, and solutions consisting of saline or water in which is dissolved another chemical such as antibiotics, antiseptics, growth factors, analgesics, and the like.

The feeding of ice particles to the injection handpiece may be in either continuous or pulsatile mode depending on the high pressure flow, which can be actuated and controlled by, for example, but not limited to, a foot switch 34 for the electrical system which controls compressor 20. Injection handpiece 18 is preferably an elongated tube in shape and of such dimensions, weight and design as to be comfortably yet tightly grasped with good operative sensitivity, and be easily maneuverable, by the operator thereof It should preferably be able to be used with one hand by either a left-handed or right-handed operator thereof The entire flow path is fabricated from materials with a low thermal conductivity and is devoid of such abrupt changes in flow cross sectional area as may lead to deposition, adherence and blockage of the path with ice. Injection handpiece 18 is preferably disposable, and is therefore preferably made from a plastic polymer, such as polycarbonate as a non-limiting example. In certain preferred embodiments, handpiece 18 is transparent.

Ice particles exit the injection outlet 32 (whose dimensions [diameter] can be between 5 and 50 mm depending on the application of the apparatus) at temperatures between −1 and 0 degrees Celsius and at a high linear velocity (between 30-100 meters/sec), under the control of an operator. The outlet is designed with dimensions (e.g., ratio of the area of the opening of outlet 32 to the smallest area of the channel within the handpiece), such that there is a pressure drop between air inlet 50 and injection outlet 32 to ensure that the desired jet (62) of particles suitable for debriding the pathological tissue and not the surrounding healthy tissue can be produced. The injection handpiece is applied to an area of tissue 40, including diseased tissue 42 and surrounding healthy tissue 44. Ice particles exiting the injection handpiece in jet 62 at approximately a 30 degree angle to the tissue will abrade the tissue and wash away both the removed tissue and the now expended abrasive ice particles, while at the same time will also provide a degree of anesthesia by chilling the surrounding tissues. Because diseased tissue 42, which can be for example, necrotic burn tissue, is more friable and more easily removed than healthy tissue, the diseased tissue 42 will be selectively abraded and removed while the surrounding healthy tissue 44 will be left undamaged. The apparatus can be used in a tub or over a surface or container that can drain or collect the melting ice and removed tissue.

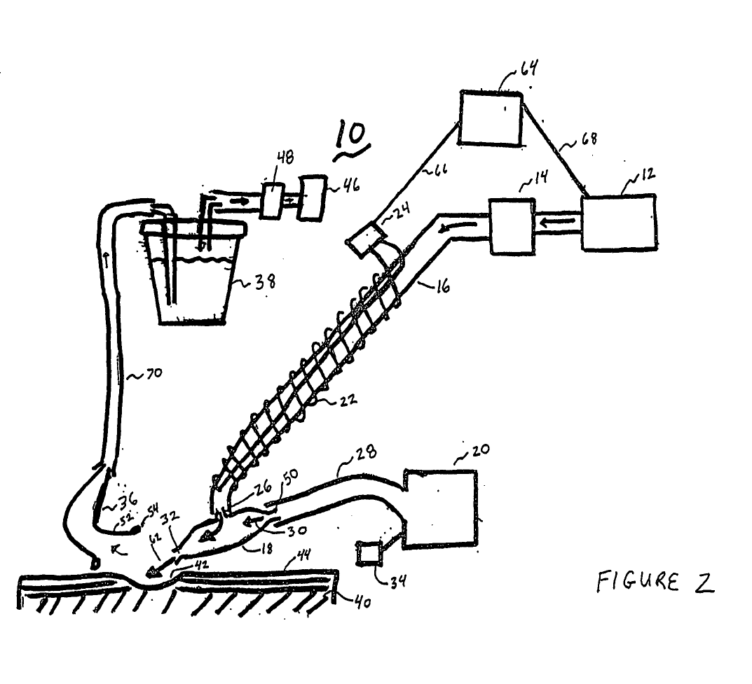

In a preferred embodiment of an apparatus for cutting, removal and debridement of biological tissue using pressurized propulsion of ice particles according to the present invention as illustrated in FIG. 2, the apparatus further includes a suction nozzle 36. This is used for aspirating the water from the melting ice along with the removed tissue fragments. Suction nozzle 36 includes a conical dome 52 with an elastic lower lip 54 that can conform to the contour of the surface to which it is applied. The suction nozzle (36) is flow connected to a vacuum source (46) via suction tubing 70 and a siphon-type suction collection container 38. Suction nozzle 36 aspirates the melting ice particles and the tissue that has been removed. Suction container 38, which is connected to suction nozzle 36 and to vacuum source 46, collects the aspirated material. This helps to prevent aerosol formation (of the materials, including tissue debris, blood and fluids, which can be hazardous to both doctor and patient) and to maintain a clean and clear operative field. The vacuum source can be a vacuum pump or the conventional vacuum system as is typically found installed in the wall of most hospital, operating and treatment rooms. A vacuum regulator 48 will preferably be connected between the vacuum source and the suction nozzle that can be adjusted so as to allow airflow sufficient in volume to prevent aerosol formation and to prevent a positive pressure build-up between the injection handpiece and the suction nozzle. This embodiment will find particular use in conjunction with methods for cosmetic peeling, for debridement of necrotic tissue, for treatment of scars and for removal of tattoos as described hereinbelow.

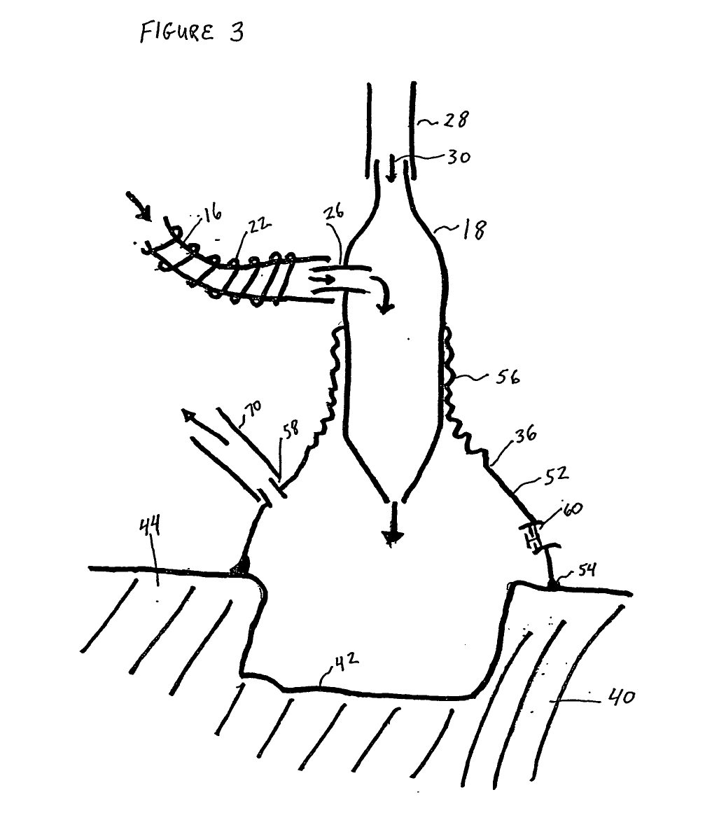

Specifically envisioned as a preferred embodiment of the present invention is a configuration suitable for use for debriding smaller or deeper areas of tissue, for example, but not limited to pressure sores and deep ulcers. In this embodiment, as illustrated in FIG. 3, suction nozzle 36 takes the form of a suction hood. Suction hood 36 has a flexible neck 56 as well as a conical dome 52 with an elastic lower lip 54. In this embodiment, injection handpiece 18 is seated within the flexible neck 56 of the suction hood (36) as is illustrated in FIG. 3. The conical dome is preferably transparent and rigid. This embodiment is configured so as to provide good maneuverability for the operator, maintaining injection handpiece 18 at a suitable angle and distance from the tissue to be debrided, while keeping the process within an enclosed housing, limiting exposure of the operator to aerosolized debris, infective material, odor and the like. Suction tubing 70 is connected to suction connection 58 on suction hood 36. Suction hood 36 also has a one way valve 60 that can allow air entry.

A number of operating parameters of the apparatus can be adjusted for various applications of the present invention. For example, the size of the ice particles can be varied from 0.001 mm to 50 mm, preferably from 0.001 mm to 15 mm, most preferably from 0.001 mm to 6 mm. The pressure generated by the high-pressure compressor to propel the ice particles from injection outlet 32 preferably range from 10-100 psi and most preferably from 20-80 psi. This will preferably generate flow rates of 10-150 grams/min of particles at linear velocities of 30-100 meter/sec. The duration of the propulsion can either be continuous or pulsatile, with pulses of from 4 to 15 seconds in duration up to one minute in total. A varied number of repeated pulses can be applied. The depth of treatment is primarily dependent upon the size of the particles and the velocity of propulsion. The smaller the particle and the faster the velocity, the greater the depth of is debridement. Temperature also plays an important role as it is the heating and melting of the ice particles that accounts for the debridement. There is a wide safety margin in terms of length of pulse and treatment, wherein treatment for longer lengths do not debride the tissues to a further, greater depth. The apparatus can be entirely under computer control of a central processor 64, which can be connected to each of the various control units. [All connections are not shown in the figures. As examples, connections to heater control 24 and ice pellet generator 12 by connections 66 and 68 respectively are shown in FIG. 1.] Central processor 64 is programmable to control all or some parameters (including, for example, temperature and presume of the main air stream, temperature of the ice particles, size of ice particles, and pulsation periods.

Preferably for removing soft necrotic tissue or for treating pressure sores, the size of the ice particles used ranges from 0.1 mm to 1 mm. In certain circumstances a mix of particle sizes can be used. The pressures used for this application range from 20-40 psi. For the debridement of burns the ice particles are 0.5-2.5 mm in size and the pressures used are 30-60 mm. For skin peeling, scar treatment and tattoo removal the size of the ice particles used is 1-6 mm and the pressures, 40-80 psi.

The above described apparatus for cutting, removal and debridement of biological tissue using pressurized propulsion of ice particles will find use primarily in conjunction with a method for cutting, removal and debridement of biological tissue using pressurized propulsion of ice particles. Such a method could be used for example for, but not be limited in its application to, a use such as the debridement of necrotic tissue, such as, for example, burn tissue.

Such a method includes the steps of generating ice pellets of a predetermined and appropriate size, delivering the ice particles to an injection, or cutting, handpiece, and propelling the ice particles toward the tissue to be cut and removed in a jet stream of a predetermined and appropriate high speed and linear velocity, so as to effect cutting and removal of the desired tissue.

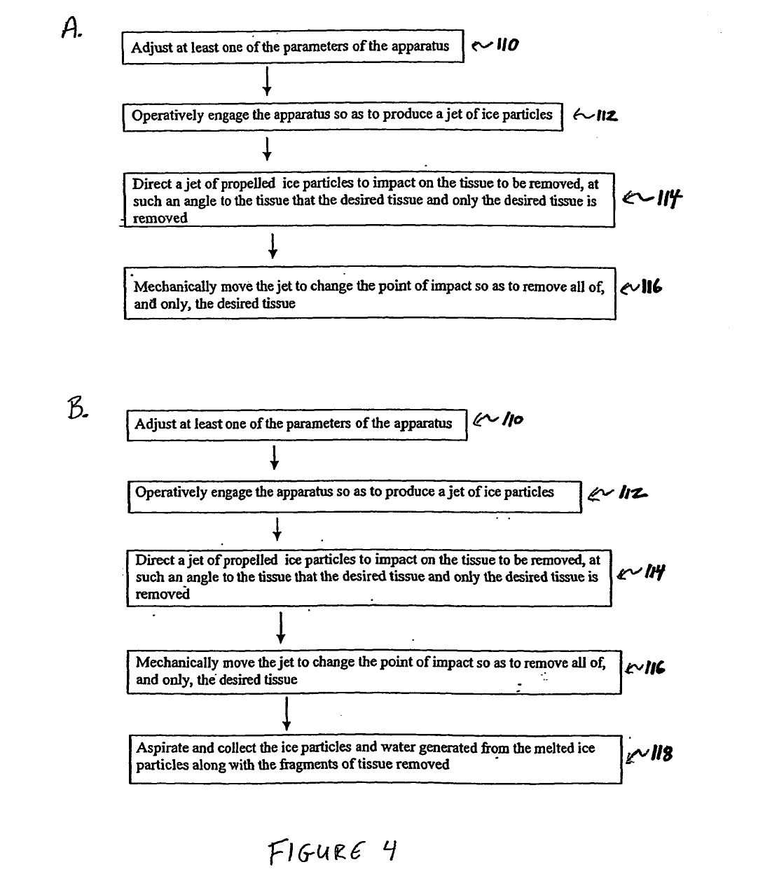

As illustrated in FIG. 4A, a specific alternate preferred embodiment of such a method includes the steps of (a) step 110—adjusting at least one of the parameters of the apparatus (10) described hereinabove, including at least one of: choosing an injection handpiece (18) with an appropriately sized injection outlet (32), selecting an appropriate size of ice particles, modifying the jet pressure, linear velocity and particle flow rate, determining whether to use continuous or pulsatile jets, determining the duration and number of the pulses and total length of application; (b) step 112—operatively engaging the apparatus so as to produce a jet of ice particles, (c) step 114—directing a jet (62) of propelled ice particles to impact on the tissue to be removed, at such an angle to the tissue that the desired tissue and only the desired tissue is removed, and (d) step 116—mechanically moving jet 62 to change the point of impact so as to remove all of, and only, the desired tissue. A further modification of the method, particularly suited to the use of a preferred embodiment of the apparatus of the present invention as illustrated in FIG. 2, is illustrated in FIG. 4B. In this modification, there is included the further step (e) step 118—of aspirating and collecting the ice particles and water generated from the melted ice particles along with the fragments of tissue removed.

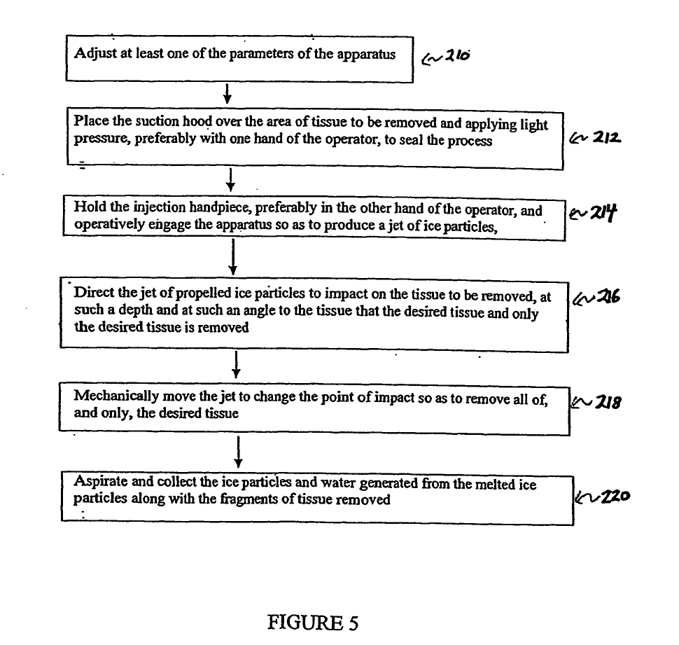

Use of the preferred embodiment of the apparatus of the present invention as illustrated in FIG. 3 involves a method (FIG. 5) that includes the following steps: (a) Step 210—adjusting at least one of the parameters of the apparatus (10) described hereinabove and illustrated in FIG. 3, including: choosing an injection handpiece (18) with an appropriately sized injection outlet (32), selecting an appropriate size of ice particles, modifying the jet pressure, linear velocity and particle flow rate, determining whether to use continuous or pulsatile jets, determining the duration and number of the pulses and total length of application, and adjusting the suction flow; (b) Step 212—placing the suction hood over the area of tissue to be removed and applying light pressure, preferably with one hand of the operator, to seal the process; (c) Step 214—holding the injection handpiece, preferably in the other hand of the operator, and operatively engaging the apparatus so as to produce a jet of ice particles, (d) Step 216—directing the jet of propelled ice particles to impact on the tissue to be removed, at such a depth and at such an angle to the tissue that the desired tissue and only the desired tissue is removed; (e) Step 218—mechanically moving the jet to change the point of impact so as to remove all of, and only, the desired tissue and (f) Step 220—aspirating and collecting the ice particles and water generated from the melted ice particles along with the fragments of tissue removed.

The use of the apparatus of the present invention using the method described herein for example for the debridement of necrotic tissues such as a burn has several advantages. These include, by selecting the appropriate values for the various parameters detailed above and adjusting the apparatus accordingly, the debridement process is limited to necrotic recesses of the field leaving the viable more elastic tissue intact. Because tissue softening, debridement and washing all are accomplished in one step, hospitalization can be shortened and fewer expensive dressings and less professional time will be consumed. The process in addition to debriding the necrotic tissue will open and drain potential pus sacs extending from the bottom of the burn crater. This drainage of these abscesses releases toxins and inflammatory mediators responsible for systemic illness, fever, weight loss, and flu-like symptoms. The pressure of the jet will open these abscesses and the melting ice irrigates their content Further, when the closed system is used the foul odor is kept contained.

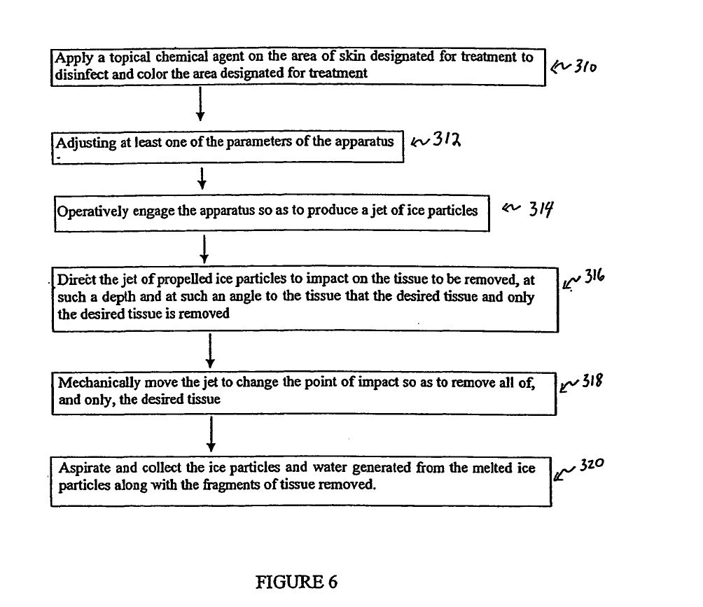

The above described apparatus will also find use in conjunction with a method for dermabrasion and chemical peeling using pressurized propulsion of ice particles. This method (FIG. 6) includes the following steps: (a) Step 310—applying a topical chemical agent on the area of skin designated for treatment to disinfect and color the area designated for treatment; (b) Step 312—adjusting at least one of the parameters of the apparatus (10) described hereinabove including: choosing an injection handpiece (18) with an appropriately sized injection outlet (32), selecting an appropriate size of ice particles, modifying the jet pressure, linear velocity and particle flow rate, determining whether to use continuous or pulsatile jets, determining the duration and number of the pulses and total length of application, and adjusting the suction flow; (c) Step 314—operatively engaging the apparatus so as to produce a jet of ice particles, (d) Step 316—directing the jet of propelled ice particles to impact on the tissue to be removed, at such a depth and at such an angle to the tissue that the desired tissue and only the desired tissue is removed; (e) Step 318—mechanically moving the jet to change the point of impact so as to remove all of, and only, the desired tissue and (f) Step 320—aspirating and is collecting the ice particles and water generated from the melted ice particles along with the fragments of tissue removed. In certain alternate preferred embodiments the step of applying a topical chemical agent on the area of skin designated for treatment to disinfect and color the area designated for treatment is omitted.

The topical chemical agent binds the most superficial keratinized layer and paints or colors it This agent is suspended in an antiseptic solution (such as povidine, or chlorhexidine in alcohol medium). The application of this agent achieves two goals. The first is that of disinfecting the skin (as is typically done before any surgical procedure). The second is that with the fist pass of the propelled jet of ice particles on the skin, the paint is removed along with the desired area for peeling. Avoiding unpainted areas prevents another pass on tissue that has already been treated. By this means, one gains control over the level and extent of peeling. However, because of the large safety margin of treatment times on treatment depth [such that depth of treatment is determined primarily by particle size and velocity] the step of applying a topical chemical agent on the area of skin designated for treatment is omitted in certain preferred embodiments.

There are several advantages of this method of peeling over the prior art methods of mechanical or laser dermabrasion and chemical peels. The ice particles cool the tissues. Cooling the tissue gives an analgesic, anaesthetic and anti-inflammatory effect, so general or partial anesthesia (essential for laser or chemical peeling) can be reduced or even avoided for superficial peeling. The melting ice particles give an irrigation effect, maintaining a sterile field and a clear view of the depth of tissue removal that has been achieved in real time. Control of depth in ‘real time’ is possible by modifying simple parameters. No active chemicals are used, eliminating the risk of hypersensitivity reactions. Further there is no risk of a systemic effect as can occur in deep chemical peels with agents such as phenol. Finally, cooling the skin increases its turgor and makes it amenable to mechanical abrasion.

Thus, it can be seen that the apparatus and methods of the present invention successfully address the shortcomings of the presently known art by providing an apparatus and methods for cutting, removal and debridement of biological tissue using pressurized propulsion of ice particles. Additional advantages to the present invention can also be seen as compared with prior art techniques using abrasive substances: use of ice particles does not cause a foreign body reaction, water or saline are inexpensive and available resources, there are no hazards to the operator due to inhalation of the particles as there is with sand blasting, and it is less aggressive than sand blasting.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An apparatus for cutting and removal of biological tissue, comprising:
a. an ice particle generator, for producing ice particles;
b. a particle delivery element, connected to said ice particle generator, for transporting said ice particles from said ice particle generator;
c. an injection handpiece, connected to said particle delivery element, said injection handpiece having an injection outlet; and,
d. a high pressure source, connected to said injection handpiece, for propelling said ice particles in a jet stream of fluid from said injection outlet, under high pressure and at high linear velocity, so as to cut and remove a desired portion of the biological tissue.

2. The apparatus of claim 1, wherein the composition of said ice particles is selected from the group consisting of frozen water, frozen saline, and solidified carbon dioxide.

3. The apparatus of claim 1, wherein said ice particles are composed of a frozen solution of a diluent containing a chemical.

4. The apparatus of claim 3, wherein said diluent is selected from the group consisting of water and saline.

5. The apparatus of claim 3, wherein said chemical is selected from the group consisting of an antibiotic, an antiseptic, an analgesic, a local anesthetic, an anticoagulant, and a growth factor.

6. The apparatus of claim 1, further comprising a low pressure compressor for moving said ice particles from said ice particle generator into said particle delivery element.

7. The apparatus of claim 1, wherein said ice particles are sucked from said particle delivery element into said injection handpiece by venturi effect.

8. The apparatus of claim 1, wherein said particle delivery element is a cannula.

9. The apparatus of claim 8, wherein said cannula is transparent.

10. The apparatus of claim 8, wherein said cannula is fabricated from a flexible material.

11. The apparatus of claim 8, wherein said cannula has an inner lumen, said inner lumen being coated with a material to prevent adherence of said ice particles to said cannula.

12. The apparatus of claim 1, further comprising a heater element for preventing said ice particles from aggregating and obstructing movement through said particle delivery element.

13. The apparatus of claim 12, wherein said heater element includes a wire, said wire being constructed from an electrically resistive material, said wire circumferentially surrounding said particle delivery element so as to apply heat to said particle delivery element.

14. The apparatus of claim 13, wherein said wire is connected to a heater control so as to maintain said applied heat at a desired temperature.

15. The apparatus of claim 1, wherein said high pressure source has a variable output pressure.

16. The apparatus of claim 15, wherein said output pressure is between 10 psi and 100 psi.

17. The apparatus of claim 15, wherein said output pressure is between 20 psi and 80 psi.

18. The apparatus of claim 1, wherein said high pressure source propels said ice particles in a stream of gas.

19. The apparatus of claim 18, wherein said gas is compressed air.

20. The apparatus of claim 18, wherein said stream of gas is maintained at a temperature of between −1 and 0 degrees Celsius.

21. The apparatus of claim 1, wherein said high pressure source propels said ice particles in a stream of liquid.

22. The apparatus of claim 1, further comprising a control switch for actuating said high pressure source.

23. The apparatus of claim 22, wherein said control switch is a footswitch.

24. The apparatus of claim 1, wherein said high pressure source is configured to be operable to continuously propel said ice particles.

25. The apparatus of claim 1, wherein said high pressure source is configured to be operable to propel said ice particles in pulses.

26. The apparatus of claim 25, wherein said pulses are between 4 and 15 seconds in duration.

27. The apparatus of claim 1, wherein said injection outlet is between 5 mm and 50 mm in diameter.

28. The apparatus of claim 1, further comprising a suction mechanism for aspirating melting ice particles and removed tissue fragments.

29. The apparatus, of claim 28, wherein said suction mechanism includes a suction nozzle.

30. The apparatus of claim 29, wherein said suction nozzle is shaped as a conical dome, said dome having an elastic lip on the distal edge of said dome that conforms to a contour of a surface against which said dome is applied.

31. The apparatus of claim 30, wherein said dome is transparent.

32. The apparatus of claim 29, wherein said suction nozzle is a suction hood having a conical dome at a distal end of said hood, said dome having an elastic lip on a distal edge of said dome that conforms to a contour of a surface against which said dome is applied, said hood having a flexible neck portion at a proximal end of said hood.

33. The apparatus of claim 32, wherein said injection handpiece and said suction hood are so configured as to place said injection handpiece within said flexible neck of said suction hood.

34. The apparatus of claim 32, wherein said suction hood further includes a one way valve configured so as to be operable to permit air entry.

35. The apparatus of claim 28, wherein said suction mechanism further includes a collection container for collecting said melting ice particles and said removed tissue fragments.

36. The apparatus of claim 1, wherein said ice particle generator includes a sizer mechanism to insure that all of said ice particles are of a predetermined and uniform size.

37. The apparatus of claim 36, wherein said predetermined size is between 0.001 mm and 50 mm.

38. The apparatus of claim 36, wherein said predetermined size is between 0.001 mm and 15 mm.

39. The apparatus of claim 36, wherein said predetermined size is between 0.001 mm and 6 mm.

40. The apparatus of claim 1, further comprising a central processor mechanism for control of at least one parameter of operation of the apparatus.

41. The apparatus of claim 40, wherein said central processor mechanism is programmable.

42. The apparatus of claim 40, wherein said at least one parameter of operation is selected from the group consisting of temperature of said fluid stream, pressure of said fluid stream, velocity of propulsion of said fluid stream, temperature of said ice particles, and size of said ice particles.

43. The apparatus of claim 1, wherein the apparatus is configured for cutting and removal of human tissue.

44. The apparatus of claim 1, wherein the apparatus is configured for cutting and removal of cutaneous tissue.

45. The apparatus of claim 1, wherein the apparatus is configured for cutting and removal of necrotic tissue.

46. The apparatus of claim 1, wherein the apparatus is configured for debriding a burn.

47. The apparatus of claim 1, wherein the apparatus is configured for debriding a pressure sore.

48. The apparatus of claim 1, wherein the apparatus is configured for performing a skin peel.

49. A method for cutting and removal of biological tissue comprising the steps of:
a. generating ice particles of a predetermined and appropriate size;
b. delivering said ice particles to an injection handpiece; and,
c. propelling said ice particles toward the biological tissue in a jet stream of a predetermined and appropriate high speed and linear velocity, so as to effect cutting and removal of a desired portion of the biological tissue.

50. A method for cutting and removal of biological tissue comprising the steps of:
a. providing an apparatus for cutting and removal of the biological tissue;
b. adjusting at least one parameter of said apparatus;
c. operatively engaging said apparatus so as to produce a jet of ice particles;
d. directing said jet of propelled ice particles to impact on the biological tissue to be removed, at such an angle to the tissue that a desired portion of the tissue and only said desired portion of the tissue is removed; and,
e. mechanically moving said jet to change a point of impact so as to remove all of, and only, said desired portion of the tissue.

51. The method of claim 50, wherein said at least one parameter is selected from the group consisting of: size of an injection outlet, size of said ice particles, pressure of said jet, velocity of said jet, pulsation of said jet, duration of said pulsation, and length of time of treatment.

52. The method of claim 50, further comprising the step of:
f. aspirating and collecting said ice particles and water generated from melting of said ice particles along with fragments of the tissue removed.

53. The method of claim 52, further comprising the steps of:
b1. placing a suction hood over an area of the tissue to be removed;
b2. applying light pressure to said suction hood to create a tight seal; and,
b3. holding an injection handpiece;
before the step of operatively engaging said apparatus so as to produce a jet of ice particles.

54. The method of claim 50, further comprising the initial step of:
applying a topical chemical agent to the tissue to disinfect and color an area designated for treatment.
